(19) United States
(12) Patent Application Publication
(10) Pub. No.: US 2019/0046482 A1
(43) Pub. Date: Feb. 14, 2019

(54) COMPOSITIONS AND METHODS FOR IMPROVING MITOCHONDRIAL FUNCTION

(71) Applicant: Mitronite Inc., Toronto (CA)

(72) Inventor: Francesco Merante, Etobicoke (CA)

(21) Appl. No.: 16/078,382

(22) PCT Filed: Feb. 23, 2017
(86) PCT No.: PCT/CA2017/050234

§ 371 Date: Aug. 21, 2018

Related U.S. Application Data
(60) Provisional application No. 62/298,715, filed on Feb. 23, 2016.

(51) Int. Cl. A61K 31/194; A61K 31/19; A61K 31/375; A61K 45/06; A61K 9/00; A61K 9/68

(52) U.S. Cl. CPC A61K 31/194; A61K 31/19; A61K 9/0058; A61K 45/06; A61K 9/0056; A61K 31/375

(57) ABSTRACT

The disclosure provides compositions comprising tricarboxylic acid cycle intermediates and at least one anti-oxidant. In one embodiment, the composition comprises pyruvic acid, citric acid and malic acid in combination with the anti-oxidant ascorbic acid. The disclosure also provides methods and uses of the compositions for improving mitochondrial function and physical recovery post-exertion.

### RELATED APPLICATIONS

This application claims the benefit of priority of U.S. provisional patent application No. 62/298,715 filed on Feb. 23, 2016, the entire contents of which are hereby incorporated by reference.

### FIELD OF THE INVENTION

The disclosure provides compositions comprising tricarboxylic acid intermediates and at least one anti-oxidant. The disclosure also provides methods and uses of the compositions for improving mitochondrial function and physical recovery post-exertion.

### BACKGROUND OF THE INVENTION

Physical and mental exertion places a demand on cellular metabolic reserves. Proteinaceous compounds such as whey protein isolate, casein or individual amino acids have been used in attempts to improve recovery. Although beneficial, these components aim to repair or augment muscle sarcolemma and only indirectly aid mitochondrial support and recovery, or improved aerobic function.

A number of different agents have been suggested to improve or augment performance and mitochondrial function in athletes. These include, but are not limited to supplementary glucose, amino acids^{2,4} (i.e., L-arginine²⁷, beta-alanine, citrulline (in the form of citrulline-malate or citrulline alone^{22,32}, glutamine⁴, taurine¹³ among other branched-chain amino acids²⁹, creatine (often in the form of pyruvate-creatine or creatine citrate¹⁵, carnitine^{38, 10}, various vitamins and antioxidants²⁵, among many others, particularly plant derived extracts or natural products (for example green tea¹², Cordyceps sinensis and yohimbine¹¹) or complex derivatives thereof concocted into fortified beverages with supplementary carbohydrates^{6,38} or protein isolates³⁷. The beneficial role of these agents has typically been investigated using high dosages of a single agent and defined exercise routines.

Additional publications have explored the augmentation of athletic performance by boosting nitric oxide levels via supplementation with L-arginine^{27, 42}, or indirectly by using an arginine precursor such as citrilluline²² which has been shown to extend time to exhaustion⁴².

Endurance and recovery has been shown to be improved by supplementing with creatine. Creatine boosts intracellular phosphor-creatine levels and can act as an energy reserve compound improving athletic performance^{7,33} when provided at therapeutically significant doses, but may contribute to increased fat and triglyceride levels in supplemented athletes¹⁷. Similarly, branched-chain amino acids have been shown to improve performance and recovery⁵, but not necessarily exercise induced muscle damage⁹.

To directly target mitochondria, the cellular powerhouses, various individual metabolic intermediates have been tested with varying degrees of success. There remains a need for improved compositions and methods for improving mitochondrial function and post-exertion recovery.

### SUMMARY OF THE INVENTION

The disclosure provides a composition comprising one or more tricarboxylic acid intermediates or salts thereof, and one or more anti-oxidants.

In one embodiment, the one or more tricarboxylic acid intermediates are selected from citric acid, malic acid, pyruvic acid, succinic acid, and/or their corresponding salts thereof. In one embodiment, the composition comprises, consists essentially of, or consists of citric acid, malic acid, pyruvic acid and succinic acid. In one embodiment, the composition comprises, consists essentially of, or consists of citric acid, malic acid and pyruvic acid.

In another embodiment, the one or more anti-oxidant agents are selected from ascorbic acid, a salt thereof, vitamin E and L-cysteine. In one embodiment, the anti-oxidant agent is ascorbic acid or a salt thereof.

In one embodiment, the composition comprises, consists essentially of, or consists of citric acid, malic acid, pyruvic acid and ascorbic acid, and optionally succinic acid.

In one embodiment, the composition comprises, consists essentially of, or consists of 2 to 4 parts by weight of pyruvic acid, 2 to 8 parts by weight of citric acid and 3 to 6 parts by weight of malic acid. In another embodiment, the composition comprises 0.5 to 4 parts by weight of ascorbic acid. Optionally, the composition comprises 0.1 to 2 parts by weight of succinic acid.

In another embodiment, the molar ratio of malic acid to pyruvic acid to citric acid to ascorbic acid is about 1:1:1:1. In one embodiment, the molar ratio of malic acid to pyruvic acid to citric acid to ascorbic acid is about 1:1:0.5:1. In another embodiment, the molar ratio of malic acid to pyruvic acid to citric acid to ascorbic acid is about 1.5:1:1:0.5. In one embodiment, the molar ratio of malic acid to pyruvic acid to citric acid to ascorbic acid is between about 1:1:1:1 and 1:1:0.5:1 or between about 1:1:1:1 and 1.5:1:1:0.5.

In another embodiment, the composition described herein comprises, consists essentially of, or consists of an effective dose of TCA cycle intermediates and an antioxidant suitable for use in a subject in need thereof. For example, in one embodiment the composition comprises from 200 to 400 mg of pyruvic acid, from 200 to 800 mg of citric acid and from 300 to 600 mg of malic acid. In one embodiment, the composition comprises from 100 to 400 mg of ascorbic acid. Optionally, the composition further comprises from 10 to 200 mg or from 50-150 mg of succinic acid.

In one embodiment, the composition comprises, consists essentially of, or consists of about 200 mg of pyruvic acid, about 300 mg of malic acid, about 200 mg of citric acid and about 100 mg of ascorbic acid. Optionally, the composition further comprises, consists essentially of, or consists of about 100 mg succinic acid. In one embodiment, the composition comprises, consists essentially of, or consists of about 400 mg of pyruvic acid, about 600 mg of malic acid, about 400 mg of citric acid and about 200 mg of ascorbic acid. Optionally, the composition further comprises, consists essentially of, or consists of about 200 mg succinic acid.

In another embodiment, the composition further comprises, consists essentially of, or consists of one or more citric acid cycle precursors, vitamins, minerals, micronutrients, electrolytes, carbohydrates, amino acids and/or proteins.

In another embodiment, the composition further comprises one or more flavouring agents.

In another embodiment, the composition further comprises one or more excipients.

In another embodiment, the composition is in the form of a liquid, a paste, a gel, a bar, a powder, a tablet or a capsule. In another embodiment, the tablet is a lozenge, a candy or a chewing gum.

In one embodiment, the compositions described herein do not include carbohydrates, such as sucrose or glucose, or protein.

The disclosure also provides a method of improving mitochondrial function in a subject in need thereof comprising administering to the subject an effective amount of a composition as disclosed herein. The disclosure further provides a use of a composition as disclosed herein for improving mitochondrial function in a subject in need thereof.

The disclosure also provides a method of decreasing cellular lactic acid in a subject in need thereof comprising administering to the subject an effective amount of a composition as disclosed herein. The disclosure further provides a use of a composition as disclosed herein for decreasing cellular lactic acid in a subject in need thereof.

The disclosure also provides a method of improving recovery following exertion in a subject comprising administering to the subject an effective amount of a composition as disclosed herein. The disclosure further provides a use of a composition as disclosed herein for improving recovery following exertion in a subject in need thereof. In one embodiment, the composition is administered or for use prior to, during and/or after exertion by the subject. In one embodiment, the composition is administered or for use within 30 minutes prior to exertion, during exertion, and/or within 30 minutes after exertion by the subject.

The disclosure also provides a method of improving peak power output in a subject comprising administering to the subject an effective amount of a composition as disclosed herein. The disclosure further provides a use of a composition as disclosed herein for improving peak power output in a subject.

The disclosure also provide a method of extending time to exhaustion for a subject performing a physical exertion comprising administering to the subject an effective amount of a composition as disclosed herein. The disclosure further provides a use of a composition as disclosed herein for extending time to exhaustion in a subject. In one embodiment, the composition is administered and/or for use in the subject prior to performing the physical exertion.

In one embodiment, the subject is a mammal, optionally a human.

In another embodiment, the composition is for use or administered prior to, during and/or after physical or mental exertion by the subject. In one embodiment, the composition is for use, or administered, prior to, during and after physical or mental exertion by the subject. In one embodiment, the composition is for use or administered within 30 minutes prior to exertion, during exertion, and within 30 minutes after exertion.

In another embodiment, the composition is administered or is for use within 2 hours, 90 minutes, 50 minutes, or 25 minutes of physical or mental exertion by the subject.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will now be described in relation to the drawings in which:

FIG. 1 shows the effect of various Krebs cycle intermediates on the rate of lactic acid production in BHK cells.

FIG. 2 shows the effect of various substrates on lactate production in C2C12 culture muscle cells. Substrate condition “all” includes 1 mM citrate, malate, pyruvate and ascorbate. C2C12 cells exposed to 1 mM glucose alone under similar conditions resulted in the cells detaching from the plates, indicative of metabolic stress.

FIG. 3 shows the increase in dead weight lifted (single heavy lift) for a test subject over a 6-month period of training sessions using a formulation comprising citric acid, malic acid, pyruvic acid and ascorbic acid as described herein

FIG. 4 shows the increase in squat weight (single heavy lift) for a test subject over a 6-month period of training sessions using a formulation comprising citric acid, malic acid, pyruvic acid and ascorbic acid as described herein.

FIG. 5 shows the increase in bench press weight (single heavy lift) for a test subject over a 6-month period of training sessions using a formulation comprising citric acid, malic acid, pyruvic acid and ascorbic acid as described herein.

FIG. 6 shows a summary of progress for single heavy lifts for a test subject over a 6-month period of training sessions using a formulation comprising citric acid, malic acid, pyruvic acid and ascorbic acid as described herein

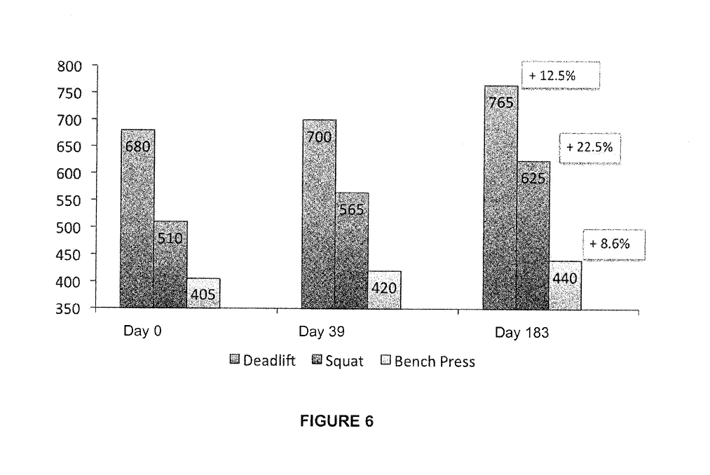

### DETAILED DESCRIPTION OF THE INVENTION

The inventors have demonstrated that the use of tricarboxylic acid (TCA) cycle intermediates in combination with an antioxidant reduces the cellular production of lactic acid. As shown in Example 1, BHK cells cultured in the presence of pyruvic, malic, citric, and succinic acids along with ascorbic acid significantly reduced the cellular levels of lactic acid. Without being limited by theory, it is believed that the combination of multiple TCA cycle intermediates and an antioxidant improves mitochondrial function and cellular performance relative to the use of individual TCA intermediates and/or antioxidants.

As shown in Examples 3-6, human subjects using a formulation comprising TCA intermediates and ascorbic acid as described herein exhibited improved performance and recovery following exertion. Without being limited by theory, the use of the compositions described herein prior to, during and/or after exertion by a subject may improve recovery by improving mitochondrial function and cellular performance. In one embodiment, the use of the compositions described herein prior to, during and after exertion by a subject is believed to facilitate the physiological recovery process and reduce the time required for physical recovery following exertion.

Accordingly, the present disclosure is directed to compositions comprising one or more tricarboxylic acid intermediates or salts thereof, in combination with at least one anti-oxidant. In some embodiments, the compositions are useful for improving mitochondrial function and/or decreasing cellular lactic acid. In some embodiments, the compositions are useful for improving recovery of a subject following exertion. In some embodiments, the compositions described herein are useful for improving performance and/or recovery following exertion in a subject in the absence of added carbohydrates such as glucose and/or added proteins.

As used herein, the term “tricarboxylic acid cycle intermediate”, “TCA intermediate” or “Krebs cycle intermediate” refers to precursors, intermediates and substrates of the citric acid cycle (Krebs cycle). Examples of tricarboxylic acid cycle intermediates include, but are not limited to, pyruvic acid, citric acid, malic acid and succinic acid and salts thereof. Other examples of tricarboxylic acid intermediates include isocitrate, oxoglutarate, fumarate, oxaloacetate and salts thereof. Accordingly, in one embodiment, the composition comprises one or more tricarboxylic acid intermediates selected from pyruvic acid, citric acid, malic acid, succinic acid, isocitrate, oxoglutarate, fumarate and oxaloacetate. In one embodiment, the composition comprises two or more tricarboxylic acid intermediates selected from pyruvic acid, citric acid, malic acid and succinic acid. In one embodiment, the composition comprises three or more tricarboxylic acid intermediates selected from pyruvic acid, citric acid, malic acid, and succinic acid. In one embodiment, the composition comprises four or more tricarboxylic acid intermediates selected from pyruvic acid, citric acid, malic acid, succinic acid, isocitrate, oxoglutarate, fumarate and oxaloacetate. In one embodiment, the composition comprises five or more, six or more, seven or more, or eight tricarboxylic acid intermediates selected from pyruvic acid, citric acid, malic acid, succinic acid, isocitrate, oxoglutarate, fumarate and oxaloacetate. In one embodiment, the composition comprises pyruvic acid, citric acid and malic acid. In one embodiment, the composition comprises pyruvic acid, citric acid, malic acid, and succinic acid. For greater clarity, reference to an organic acid such as, but not limited to, pyruvic acid, citric acid, malic acid, and/or ascorbic acid, as used herein includes any salts thereof, such as, but not limited to pyruvates (e.g. calcium pyruvate), citrates (e.g. calcium citrate), malates (e.g. sodium malate) and/or ascorbates.

In one embodiment, the compositions are in a dosage form that provides an effective amount of the one or more tricarboxylic acid cycle intermediates or salts thereof and/or antioxidants as described herein. As used herein, the term “effective amount” is an amount of a composition, or a component of a composition, effective, at dosages and for periods of time necessary to achieve the desired result. For example, an effective amount of a substance may vary according to factors such as the physical state, age, sex, and weight of the individual, and the ability of the substance to elicit a desired response in the individual. In one embodiment, the compositions described herein include an effective amount of a combination of TCA intermediates and an antioxidant such as to improve mitochondrial function and/or decrease cellular lactic acid.

Terms of degree such as “about”, “substantially”, and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

In one embodiment, the composition comprises pyruvic acid or a salt thereof. Pyruvate, the end product of glycolysis, is decarboxylated and enters the Krebs cycle in the form of acetyl co-enzyme A. This two carbon derivative, interacts with oxaloacetate (the last 4-carbon intermediate of the cycle) to regenerate citrate. Thus, pyruvic acid may be provided in the disclosed compositions to aid in the replenishment of citrate in the citric acid cycle by way of the formation of acetyl-Co-enzyme A. Pyruvic acid also participates in anaplerotic reactions via the conversion to oxaloacetate which can then be a substrate for the generation of aspartate which is a precursor for the essential amino acids methionine, threonine, isoleucine, and lysine.

Without being bound by theory, additional desirable effects of including pyruvate in the disclosed compositions may include augmented utilization of excess body weight and fat mass, likely by the stimulation of aerobic metabolism, although the mechanism of this effect has not been fully elucidated^{8,16,28,30}. Additional beneficial effects may also include improved mood states of fatigue and vigor scores¹⁶. Further, providing additional pyruvate during and post-exertion may maintain the pyruvate dehydrogenase complex (PDH) in an enzymatically active state for longer periods, augmenting recovery and post exertion lactate utilization²⁴.

Accordingly, in one embodiment, the compositions comprise an effective amount of pyruvic acid or a salt thereof. In one embodiment, an effective amount of pyruvic acid is an amount providing an effective blood concentration of pyruvic acid of between about 100-500 micromolar, optionally between about 100-400 micromolar, 150-500 micromolar, 300-500 micromolar, 150-400 micromolar, 150-300 micromolar, 150-250 micromolar or about 200 micromolar. One skilled in the art would appreciate that body mass, blood volume and degree of exertion or age may influence the required effective concentration. For example, a 70 kg individual with an approximate blood volume of 5 liters can achieve an effective blood concentration of pyruvic acid with a dose of about 50-1000 mg, about 100-500 mg, 100-300 mg, 200-400 mg, 150-250 mg or about 200 mg of pyruvic acid. In one embodiment, the compositions described herein comprise about 100 mg, 150 mg, 200 mg, 250 mg, 300 mg, 350 mg, 400 mg, 450 mg, 500 mg, 550 mg, 600 mg or 650 mg of pyruvic acid. In one embodiment, the pyruvic acid is calcium pyruvate.

In another embodiment, the composition comprises citric acid or a salt thereof. Citric acid participates in the citrate mediate reaction which includes a dehydration step to form cis-aconitate which can be decarboxylated. Nicotinamide dinucleotide (NAD) is simultaneously reduced to the high energy intermediate NADH.

Citrate is transported into the mitochondria and participates directly in the citric acid cycle. It also participates in anaplerotic reactions by way of its conversion to acetyl-CoA, which is involved in fatty acid biosynthesis, and by its conversion to isocitrate and oxoglutarate. Oxoglutarate is a required and necessary precursor for the formation of glutamate and associated amino acids, for example proline. Without being bound by theory, complementing these anaplerotic effects with citric acid improves rehydration post exercise and blood buffering capacity, thereby improving athletic recovery upwards of 16 hours post training³⁴. Citrate supplementation alone has been shown to confer improvement in swimming performance for 200 m events²⁶.

Accordingly, in one embodiment, the composition comprises an effective amount of citric acid or a salt thereof. In one embodiment, an effective amount of citric acid is an amount providing an effective blood concentration of citric acid of between about 50-500 micromolar, optionally between about 100-500 micromolar, 100-400 micromolar, 75-250 micromolar, 150-400 micromolar, 150-300 micromolar, 150-250 micromolar or about 200 micromolar. One skilled in the art would appreciate that body mass, blood volume and degree of exertion or age may influence the required effective concentration. In one embodiment, a 70 kg individual with an approximate blood volume of 5 liters can achieve an effective blood concentration of citric acid with a dose of approximately 50-2000 mg, optionally 50-1500 mg, 75-1000 mg, 100-800 mg, 200-700 mg, 200-600 mg, 200-500 mg, 150-250 mg or approximately 200 mg citric acid. In one embodiment, the compositions described herein comprise about 100 mg, 150 mg, 200 mg, 250 mg, 300 mg, 350 mg, 400 mg, 450 mg, 500 mg, 550 mg, 600 mg, 650 mg, 700 mg, 750 mg or 800 mg of citric acid. In one embodiment, the citric acid is calcium citrate.

In another embodiment, the composition comprises malic acid or a salt thereof. Malic acid is an oxaloacetate replenishing metabolite which is critical and necessary for regenerating citrate via its combination with acetyl-CoA. During this dehydrogenation reaction, malate dehydrogenation generates one molecule of NADH which can be subsequently used by oxidative reaction to produce ATP. Similarly, malate dehydrogenation to oxaloacetate produces the precursor required for the production of aspartate and associated amino acids. Without being bound by theory, malate has been shown to improve physical stamina in mice during a forced swimming protocol⁴¹. Also malate in combination with creatine exhibited significant ergogenic effects in sprinters whereby increased anaerobic and morphological indices were evaluated³⁶.

Similarly, the combination of malate with citrulline improves athletic performance and relives post-exercise induced muscle soreness²², improves stamina, reduces lactate, improves resistance performance, and reduces physical damage and promotes aerobic energy production^{2,39,40,41}.

Accordingly, in one embodiment, the compositions comprise an effective amount of malic acid or a salt thereof. In one embodiment, the malic acid is greater than 50%, 75%, 80%, 90%, 95%, or 99% L-malic acid. In one embodiment, an effective amount of malic acid is an amount providing an effective blood concentration of malic acid of about 1-100 micromolar, 1-50 micromolar, 1-25 micromolar, 1-20 micromolar, 2-15 micromolar, 3-15 micromolar, 5-15 micromolar or about 10 micromolar. One skilled in the art would appreciate that body mass, blood volume and degree of exertion or age may influence the required effective concentration. In one embodiment, a 70 kg individual with an approximate blood volume of 5 liters can achieve an effective blood concentration of malic acid with a dose of about 50-1500 mg, optionally 100-1500 mg, 200-1200 mg, 250-1000 mg, 250-800 mg, 250-650 mg, 300-600 mg or approximately 450 mg malic acid. In one embodiment, the composition disclosed herein comprises about 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, or 850 mg malic acid.

In another embodiment, the composition comprises succinic acid or a salt thereof. Succinate conversion to fumarate directly contributes to the reduction of FAD to FADH which may be used to produce ATP by the electron transport chain, in a manner similar to the conversion of malic acid to oxaloacetate to produce NADH from NAD. These molecules provide the reducing powder necessary to produce the intermediates and play pivitol roles in oxidative metabolism. Blood concentrations of succinic acid have been shown to be approximately 23.5 micromolar⁴³.

In one embodiment, an effective amount of succinic acid is an amount providing an effective blood concentration of succinic acid of about 5-100 micromolar, optionally between about 5-80 micromolar, 8-50 micromolar, 10-50 micromolar, 15-40 micromolar, 10-35 micromolar, 15-35 micromolar or about 15 micromolar. One skilled in the art would appreciate that body mass, blood volume and degree of exertion or age may influence the required effective concentration. In one embodiment, a 70 kg individual with an approximate blood volume of 5 liters can achieve an effective blood concentration of succinic acid with a dose of about 5-200 mg, optionally 10-150 mg, 15-150 mg, 50-150 mg, 75-125 mg, 20-40 mg, 10-40 mg, 80-140 mg or about 100 mg succinic acid. In one embodiment, the compositions disclosed herein comprise about mg succinic acid.

In yet another embodiment, the composition comprises each of pyruvic acid, citric acid and malic acid or salts thereof. In another embodiment, the composition comprises each of pyruvic acid, citric acid, malic acid and succinic acid or salts thereof. Without being bound by theory, collectively pyruvic acid, citric acid and malic acid and optionally succinic acid, complement one another and augment and/or aid mitochondrial performance and foster aerobic energy production. In addition, these intermediates can concurrently participate in anaplerotic reactions which provide the necessary biosynthetic carbon skeleton intermediates required to replenish used or exhausted metabolites required for cellular maintenance and repair.

In one embodiment, the composition comprises 2 to 4 parts by weight of pyruvic acid, 2 to 8 parts by weight of citric acid, and 3 to 6 parts by weight of malic acid. In one embodiment, the composition comprises about 200-400 mg pyruvic acid, 200-800 mg citric acid, 200-600 mg of malic acid and optionally 50-150 mg of succinic acid.

The disclosed compositions further comprise an anti-oxidant. As used herein, the term “anti-oxidant” refers to a substance that inhibits the oxidation of other molecules such as by donating electrons to free radicals or other oxidizing agents. Examples of anti-oxidants include, but are not limited to ascorbic acid or a salt thereof, vitamin E and L-cysteine. In a preferred embodiment, the anti-oxidant is ascorbic acid.

In one embodiment, the disclosed compositions comprise ascorbic acid or a salt thereof. Without being bound by theory, ascorbic acid provides an anti-oxidant role for both the collective intermediates and as a water soluble cellular anti-oxidant. It is also used to quell oxidative free radicals particularly during the re-establishment of oxidative metabolism following a period of glycolytically derived ATP production. This anti-oxidant has been shown to minimize myocyte damage during periods of intense and exhaustive exercise and preserve mitochondrial function, reduce a marker of oxidative damage as exemplified by carbonyl content and increase anti-oxidant capacity²⁵.

Ascorbic acid may also contribute to mitochondrial energetics in a manner independent of its role as an antioxidant and contribute to the re-energizing of cellular respiration and mitochondrial performance during periods of aerobic stress⁴⁴. Ascorbic acid may also exert an additoinal effect by maintaining cellular redox statue.

Accordingly, in one embodiment, the composition comprises an effective amount of ascorbic acid or a salt thereof. In one embodiment, an effective amount of ascorbic acid is an amount providing an effective blood concentration of ascorbic acid of 10-2000 micromolar, optionally 50-1000 micromolar, 50-750 micromolar, 50-500 micromolar, 75-250 micromolar, 20-100 micromolar, 50-100 micromolar, 60-90 micromolar or about 80 micromolar, although one skilled in the art would appreciate that body mass, blood volume and degree of exertion or age may influence the required effective concentration. In one embodiment, a 70 kg individual with an approximate blood volume of 5 liters can achieve an effective blood concentration of ascorbic acid with a dose of 50-1500 mg, optionally 50-750 mg, 50-500 mg, 75-250 mg, or approximately 100 mg ascorbic acid. In one embodiment, the composition described herein comprise about 50 mg, 75 mg, 100 mg, 125 mg, 150 mg, 200 mg, 250 mg, 300 mg, 350 mg, 400 mg, 450 mg, or 500 mg ascorbic acid.

In one embodiment, the composition comprises 2 to 4 parts by weight of pyruvic acid or a salt thereof, 2 to 8 parts by weight of citric acid or a salt thereof and 2 to 6 parts by weight of malic acid or a salt thereof and 0.5 to 1.5 parts by weight of ascorbic acid or a salt thereof. In another embodiment, the composition comprises about 100-300 mg pyruvic acid or a salt thereof, 100-300 mg citric acid or a salt thereof, 200-400 mg of malic acid or a salt thereof and 50-150 mg of ascorbic acid or a salt thereof.

In one embodiment, the compositions described herein have about an equimolar amount TCA cycle intermediates and optionally an equimolar amount of an antioxidant such as ascorbic acid. In one embodiment, the molar ratio of malic acid to citric acid to pyruvic acid to ascorbic acid is about 1:1:1:1. In one embodiment, the compositions described herein comprise succinic acid and the molar ratio of malic acid to citric acid to pyruvic acid to ascorbic acid to succinic acid is from about 1:1:1:1:1 to 1:1:1:1:0.05. In one embodiment, the compositions described herein comprise succinic acid and the molar ratio of malic acid to citric acid to pyruvic acid to ascorbic acid to succinic acid is from about 1:1:1:1:1 to 1:1:1:1:0.5.

In one embodiment, the molar ratio of malic acid to citric acid to pyruvic acid to ascorbic acid is about 1.5:1:1:0.5. In one embodiment, the compositions described herein comprise succinic acid and the molar ratio of malic acid to citric acid to pyruvic acid to ascorbic acid to succinic acid is from about 1.5:1:1:1:1 to 1:1:1:0.5:0.05 or from about 1.5:1:1:1:1 to 1.5:1:1:0.5:0.5.

In other embodiments, the disclosed compositions comprise additional components to further enhance or augment the effectiveness of the composition. Examples of additional components useful in the disclosed compositions include, but are not limited to, additional citric acid cycle precursors, intermediates and substrates, vitamins, minerals, micronutrients, electrolytes, carbohydrates, amino acids and/or proteins. Examples of proteins include, but are not limited to, whey isolate, casein, soya derived protein, etc.

In other embodiments, the disclosed compositions further comprise a flavouring agent. Examples of flavouring agents are well known in the art.

The compositions disclosed herein may be formulated into compositions for administration to subjects and/or use in subjects in a biologically compatible form suitable for administration in vivo. In particular, the disclosed compositions may comprise a pharmaceutically acceptable excipient or carrier. Suitable pharmaceutically acceptable excipients or carriers include essentially chemically inert and nontoxic compositions that do not interfere with the effectiveness of the biological activity of the composition. Suitable vehicles are described, for example, in Remington's Pharmaceutical Sciences (Remington's Pharmaceutical Sciences, 20^{th} ed., Mack Publishing Company, Easton, Pa., USA, 2000). On this basis, the compositions include, albeit not exclusively, solutions of the substances in association with one or more pharmaceutically acceptable vehicles or diluents, and contained in buffered solutions with a suitable pH and iso-osmotic with the physiological fluids.

Examples of suitable excipients include, but are not limited to, water, saline solutions, glycerol solutions, ethanol, N-(1(2,3-dioleyloxy)propyl)N,N,N-trimethylammonium chloride (DOTMA), diolesylphosphotidyl-ethanolamine (DOPE), and liposomes. Such compositions should contain an effective amount of the various components, together with a suitable amount of excipient so as to provide the form for direct administration to the subject.

The disclosed compositions are optionally in the form of a liquid (for example, a beverage), a paste, a gel, a bar, a powder, a tablet or a capsule. Examples of tablets include, but are not limited to, a lozenge, a candy, a soft-chew or a chewing gum. A tablet or capsule optionally contains a full dose or less than a full dose (for example, a half dose) of the composition.

Compositions further include, without limitation, lyophilized powders or aqueous or non-aqueous sterile injectable solutions or suspensions, which may further contain additional anti-oxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions. Proteins may be supplied, for example but not by way of limitation, as a lyophilized powder which is reconstituted with sterile water or saline prior to administration to the patient.

In another embodiment of the disclosure, the compositions are a component of food, or an additive to food.

A sterile form of the preparation in a liquid format can be envisioned to be used therapeutically in a clinical setting to provide metabolic support in emergency, acute or medially prescribed cases, for example during trauma or lactic acidosis. It is anticipated that an intravenous sterile preparation at a physiological pH value can be prepared and administered as specified by a medical professional.

In one embodiment, the composition may comprise hypermellose, microcrystalline cellulose and/or magnesium stearate. In one embodiment, the composition comprises trace amounts (e.g. less than 1%, less than 0.5%, less than 0.1%, less than 0.05% or less than 0.01%) of hypermellose, microcrystalline cellulose and/or magnesium stearate. In one embodiment, the composition consists essentially of pyruvic acid, citric acid, malic acid, and ascorbic acid and has trace amounts of excipients, carriers or compounds such as hypermellose, microcrystalline cellulose and/or magnesium stearate.

In a preferred embodiment, the composition is provided in a dosage form suitable for oral human consumption. In one embodiment, the dosage form is a sachet, capsule or tablet that comprises, consists essentially of, or consists of malic acid, pyruvic acid, citric acid and ascorbic acid. In one embodiment, a dosage comprises between about 500 mg and 1500 mg, optionally between about 750 mg and 1000 mg, or about 800 mg.

In one embodiment, the dosage form comprises, consists essentially of, or consists of 150-250 mg of citric acid, 250-350 mg of malic acid, 150-250 mg of pyruvic acid and 50-150 milligrams of ascorbic acid. In one embodiment, the dosage form comprises, consists essentially of, or consists of about 200 mg citric acid, about 300 mg malic acid, about 200 mg pyruvic acid and about 100 milligrams of ascorbic acid.

In one embodiment, the composition or dosage form comprises, consists essentially of, or consists of 150-250 mg of calcium citrate, 250-350 mg of L-malic acid, 150-250 mg of calcium pyruvate, and 50-150 milligrams of ascorbic acid. In one embodiment, the composition or dosage form comprises, consists essentially of, or consists of 200 mg of calcium citrate, 300 mg of L-malic acid, 200 mg of calcium pyruvate, and 100 milligrams of ascorbic acid.

A number of supplements marketed for use by athletes or to be consumed prior to, during, or after exertion contain carbohydrates such as glucose or sucrose and/or proteins. One advantage of the compositions described herein is that they are particularly effective for improving recovery in the absence of added carbohydrates and/or proteins. Furthermore, some subjects may not wish to consume high levels of carbohydrates and/or proteins but still wish to improve performance and/or recovery following exertion. Accordingly, in one embodiment, the composition described herein does not contain added carbohydrates such as sucrose, dextrose or glucose. In one embodiment, the composition described herein does not contain added protein.

As shown in Example 6, the use of a composition as described herein in the absence of added carbohydrates and/or protein may result in improved performance and/or physical recovery.

### Methods and Uses

The present disclosure is also directed to methods and uses of the disclosed compositions, as well as compositions for the uses described herein.

In particular, the disclosure provides a method of improving mitochondrial function in a subject in need thereof, comprising administering to the subject an effective amount of the disclosed compositions. The disclosure also provides use of the disclosed compositions for improving mitochondrial function in a subject in need thereof as well as a composition as described herein for improving mitochondrial function in a subject in need thereof.

As used herein, the term “improving mitochondrial function” includes improving mitochondrial function in a subject by at least 2, 3, 4, 5, 10, 25, 50, 100 or 200%. Mitochondrial function may be assessed by any method known in the art. For example, mitochondrial function may be assessed by oxygen consumption respiration studies such as by using a Clark oxygen electrode.

In one embodiment, mitochondrial function is assessed by monitoring the amount or rate of cellular lactic acid production. Here, a decrease in the amount of lactic acid or a decrease in the rate lactic acid production indicates improved mitochondrial function. Optionally, the amount or rate of lactic acid production is decreased by at least 2, 3, 4, 5, 10, 25, 50, 100 or 200% relative to controls who do not use the compositions described herein.

The disclosure also provides a method of decreasing cellular lactic acid in a subject in need thereof, comprising administering to the subject an effective amount of the disclosed compositions. The disclosure also provides a use of the disclosed compositions for decreasing cellular lactic acid in a subject in need thereof as well as a composition as described herein for decreasing cellular lactic acid in a subject in need thereof.

Various methods of assaying for lactic acid are known in the art, such as the method described in Example 1. Cellular lactic acid may also be assayed using fluoremetric detection⁴⁷, colorimetric assessment⁴⁸, or chemical conversion to acetaldehyde⁴⁹. In one embodiment, cellular lactic acid is decreased by at least 2, 3, 4, 5, 10, 25, 50, 100 or 200% relative to controls who do not use the compositions described herein.

The disclosure further provides a method of increasing or improving recovery from physical exertion in a subject in need thereof, comprising administering to the subject an effective amount of the disclosed compositions. The disclosure also provides use of the disclosed compositions for increasing or improving physical recovery from exertion in a subject in need thereof. Also provided is a composition as described herein for increasing or improving recovery from physical exertion.

As used herein, the term “increasing physical recovery” or “improving physical recovery” refers to any improvement in a subject's physical state post physical exertion. “Increasing physical recovery” can include, but is not limited to, decreasing fatigue, improving energy, decreasing muscle fatigue, decreasing muscle soreness, decreasing time of return to resting heartbeat and resting respiration and increasing endurance. In one embodiment, a measure of physical recovery is increased by at least 2, 3, 4, 5, 10, 25, 50, 100 or 200% relative to physical recovery in the absence of the compositions described herein. In one embodiment, “physical recovery” refers to a period following exertion characterized by a return towards a similar physiological state for a subject relative to the physiological state of the subject prior to exertion. In one embodiment, “physical recovery” for a subject following exertion includes the return of peak or optimum performance, such as power output. In one embodiment, “physical recovery” includes the restoration of musculoskeletal function following the stresses and forces of exertion, the restoration of energy stores depleted by exertion, and/or a reduction in cellular and/or blood levels of lactic acid/lactate. In one embodiment, “improving physical recovery” refers to reducing the time needed for a subject to return to a similar physiological state and/or performance level relative to the physiological state and/or performance level of the subject prior to exertion.

As used herein, the term “physical exertion” refers to any activity such as exercise, which causes the subject to become physically tired or fatigued. Physical exertion includes, but is not limited to, aerobic activity, anaerobic activity (for example, strength training), endurance training, stretching and cardiopulmonary exercising. Examples of activities that could lead to physical exertion include walking, running, swimming, dancing, climbing and participating in team or individual sports. In one embodiment, physical exertion includes an increase expenditure of energy by skeletal muscles relative to when a subject is at rest. Physiological characteristics of exertion may include increased heart rate, increased respiration rate, sweating, increased cellular and/or blood levels of lactic acid/lactate, and/or an increase in body temperature.

Other methods and uses of the disclosed compositions include, but are not limited to, augmenting recovery of mitochondrial aerobic capacity and concurrent performance, increasing time to anaerobic threshold¹⁹ and delaying in time to fatigue. In other embodiments, the compositions may be useful for hastening physical recovery, minimizing soreness and/or fostering repair of micro-damage^{1,20}. The compositions may also be useful for increasing mental alertness, augmenting capacity during the aged state, and improving fertility in cases where mitochondrial energetics are responsible for the reduced fertility state³.

The compositions disclosed herein may be administered to, or used in, living organisms including humans, and animals. The term “subject” or “animal” as used herein refers to any member of the animal kingdom, preferably a mammal, more preferably a human.

Administration of an “effective amount” of the composition is defined as an amount effective, at dosages and for periods of time necessary to achieve the desired result. For example, an effective amount of a composition or a component of a composition may vary according to factors such as the disease state, age, sex, and weight of the individual, and the ability of the composition or component to elicit a desired response in the individual. The dosage regime may be adjusted to provide the optimum therapeutic response. For example, several divided doses may be administered daily or the dose may be proportionally reduced as indicated by the exigencies of the therapeutic situation.

The disclosed compositions can be administered or use, prior to, during and/or after physical or mental exertion by the subject. In one embodiment, the compositions disclosed herein are administered within 2 years, 90 minutes, 1 hour, 50 minutes, 30 minutes or 25 minutes of physical or mental exertion by the subject.

In one aspect, use of a composition as described herein, prior to, during and after physical exertion is particularly effective at improving physical recovery. In one embodiment, the same composition is used prior to, during and after physical exertion in order to improve recovery following exertion. In contrast, subjects previously may have used different compositions prior to, during or after exertion in order to try and improve physical recovery. In one embodiment, the same dose of the composition is for use or administered to a subject prior to, during and after a period of exertion.

Accordingly, in one embodiment, there is provided a method for improving physical recovery in a subject following a period of exertion, the method comprising:

administering a first dose of a composition as described herein to the subject prior to the period of exertion, optionally 30 minutes, 15 minutes, 10 minutes or 5 minutes prior to exertion;

administering a second dose of a composition as described herein to the subject during the period of exertion; and

administering a third dose of a composition as described herein to the subject after the period of exertion, optionally within 30 minutes, 15 minutes, 10 minutes or 5 minutes after the period of exertion.

Also provided is a use of a composition for improving physical recovery in a subject following a period of exertion, comprising use of a first dose of a composition as described herein prior to exertion, optionally 30 minutes, 15 minutes, 10 minutes or 5 minutes prior to exertion, use of a second dose of a composition as described herein during the period of exertion, and use of a third dose of a composition as described herein, optionally within 30 minutes, 15 minutes, 10 minutes or 5 minutes after the period of exertion.

In one embodiment, the first, second and third dose comprise, consist essentially of, or consist of citric acid, malic acid, pyruvic acid and ascorbic acid. In one embodiment, the first, second and third dose are the same.

In one embodiment, each dose comprises between about 500 mg and 7500 mg, between about 800 mg and 6000 mg, between about 800 mg and 5000 mg, between about 800 and 4800 mg, between about 800 and 4000 mg, between about 800 mg and 3200 mg, between about 800 and 2400 mg, between about 800 and 1600 mg or about 800 mg of a composition as described herein.

In one embodiment, each dose comprises one or more dosage forms, such as a capsule, tablet or other form suitable for ingestion, that comprises, consists essentially of, or consists of 150-250 mg of citric acid, 250-350 mg of malic acid, 150-250 mg of pyruvic acid and 50-150 milligrams of ascorbic acid. In one embodiment, each dose comprises, consists essentially of, or consists of about 200 mg of citric acid, about 300 mg of malic acid, about 200 mg of pyruvic acid and about 100 milligrams of ascorbic acid.

The following non-limiting examples are illustrative of the present disclosure:

### EXAMPLE 1

The effect of various Krebs cycle intermediates was tested in a cell culture model utilizing primary BHK cells freshly seeded to permit confluency at the time of testing. Test substrates prepared in phosphate buffered saline to buffer pH changes (n=3) were added to the cultures. The protocol was effectively a modification of that presented by Merante et al., 1998¹⁸, and lactic acid concentration was determined essentially as presented by the Boehringer Mannheim lactate assay (http://www.r-biopharm.com/wp-content/uploads/4050/Lactic-acid-L_EN_10139084035_2013-03.pdf).

In brief, the cell culture supernatant was removed and the cells washed twice with PBS and subsequently maintained in PBS to deplete intracellular glycogen stores. Following the incubation, the test substrates were added to the cultures. The concentration of each was as follows: 1 mM glucose, 0.5 mM glucose+0.5 mM malic acid, 0.5 mM glucose+0.5 mM citric acid, 0.5 mM glucose+0.5 mM pyruvic acid, 0.5 mM glucose+0.5 mM succinic acid, 0.5 mM glucose+0.5 mM ascorbic acid and a combination of substrates in the absence of glucose (0.2 mM each of pyruvic, malic, citric, succinic and ascorbic acids). The substrates were incubated for 30 minutes with the cells and post-incubation, the culture supernatants were retrieved for enzymatic lactate determination. The protein content was established by standard techniques (Bradford assay) to calculate the rate of lactic acid production per condition tested.

The trend was for lactic acid concentration to decrease upon incubation of cells with a Kreb cycle substrate, even in the presence of glucose. However, the observed decrease in lactic acid was significant (*) only for test substrates comprising glucose+succinic acid (p=0.03), and the combination of pyruvic, malic, citric, succinic and ascorbic acids in the absence of glucose (p=0.016) by T-test analysis. Remarkably, the combination of pyruvic, malic, citric, succinic and ascorbic acids showed the greatest reduction in lactic acid.

BHK cells are an immortalized cell line and are highly glycolytic. Thus, in a primary cell system or whole organism, the trends and significance of metabolic augmentation may be more pronounced. Without being bound by theory, these results show that key metabolic intermediates may foster cellular performance enhancement via improved mitochondrial function as exemplified by a reduction of lactate, a key indicator of lowered flux through the mitochondrial citric acid cycle and respiratory chain¹⁸. In particular, the combination of pyruvic, malic, citric, succinic and ascorbic acids appears to have a significant effect on improving mitochondrial function and lowering levels of lactic acid. In addition to its role as an antioxidant and possible role in energizing mitochondria, ascorbic acid may also contribute a dietary role in fostering the absorption of iron from the diet and augmented iron metabolism which contributes to improved tissue oxygenation⁵⁰.

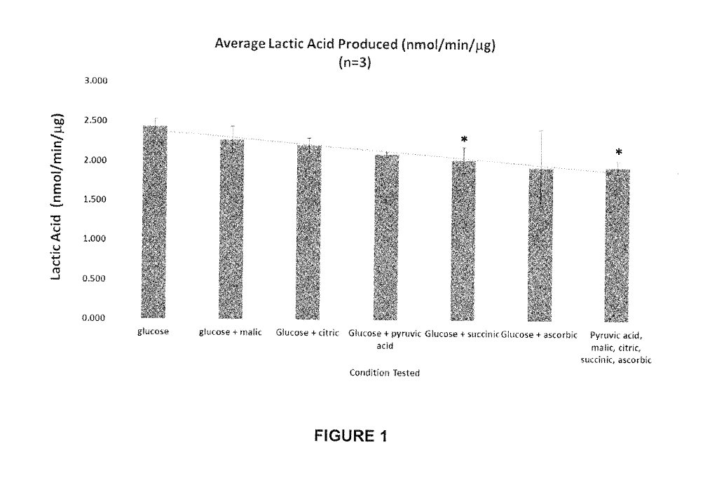

As shown in FIG. 1, when Kreb Cycle substrates were added to the cells, even in the presence of glucose, the trend was to decrease the production of lactic acid, suggesting a continued and/or an improved flux through the mitochondrial citric acid cycle and/or the mitochondrial respiratory chain.

### EXAMPLE 2

### Materials and Methods

C2C12 muscle cells were grown to confluency in six-well plates containing minimal essential media (MEM)+10% bovine serum. The cells were washed two times with phosphate buffered saline (PBS) and then incubated in PBS at 37° C. for 30 minutes to deplete intracellular glycogen. Following incubation, the PBS was removed and replaced with 1 mL of each respective substrate (n=6 for each condition) at 1 mM. Conditions marked as “all” included malate, pyruvate, citrate and ascorbate all at 1 and optionally glucose at 1 mM. Following an incubation period of 30 minutes at 37° C., metabolism was halted by the addition of 50 μL of 1.6 M perchloric acid and the supernatant was removed and immediately frozen.

The quantity of lactate produced in the culture supernatant during the incubation period was determined enzymatically and correlated to the total cellular protein, to determine the rate of lactate production (nmol/min/μg).

### Results

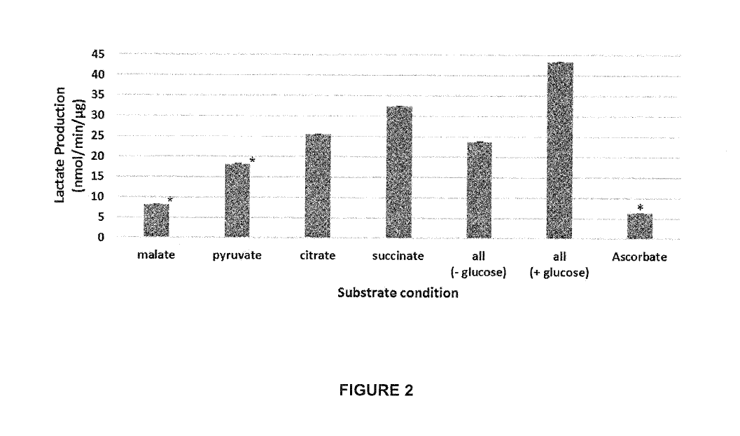

As shown in FIG. 2, significant lactate production differences (*p<0.05 by t-test relative to all+glucose) were observed for malate, pyruvate and ascorbate substrates. This indicates that the aforementioned components modulate the lactate levels and most importantly, even in the presence of glucose, the combination of these components as demonstrated by the All-condition cause a reduction of lactate suggesting augmented flux through the mitochondrial citric acid cycle and /or oxidative metabolism. Exposing C2C12 cells to glucose alone under the same conditions but without 1 mM malate, pyruvate, citrate and ascorbate resulted in the cells becoming detached from the culture plate and showing signs of metabolic stress including cell death.

### EXAMPLE 3

A study was conducted to assess the effect of a formulation of tricarboxylic acid (TCA) cycle intermediates and ascorbic acid on performance during a 6 month period for a high performance athlete. Use of the formulation was observed to improve recovery and peak power output.

### Materials and Methods

The subject was a 26 year old male active for 11 years as a power lifter. The subject was provided with capsules comprising citric acid, malic acid, pyruvic acid and ascorbic acid. Each capsule contained 200 milligrams of citric acid, 300 milligrams of malic acid, 200 milligrams of pyruvic acid and 100 milligrams of ascorbic acid.

The performance of the subject over a 6 month period was assessed for dead lift, squat and bench press. No changes to the subject's diet or use of nutritional supplements were made during the 6 month period other than the use of the formulation described above. Auto-regulation was used to very work-out intensity and volume.

3 doses (capsules) of approximately 800 mg each were consumed by the subject 15 minutes prior to each training session, intra-training as needed and immediately post-training.

### Results

### Dead Lift

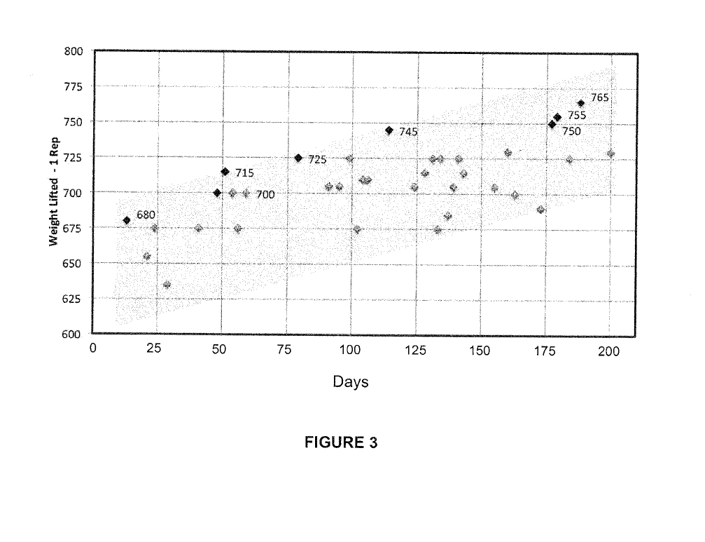

The subject completed 74 dead lift sessions out of 187 possible training days averaging 2.8 dead life sessions per week. 36 of those sessions worked up to a heavy single lift resulting in 8 new personal records. As shown in FIG. 3, the subject displayed a consistent improvement in dead lift performance over the 6-month period using the formulation.

### Squats

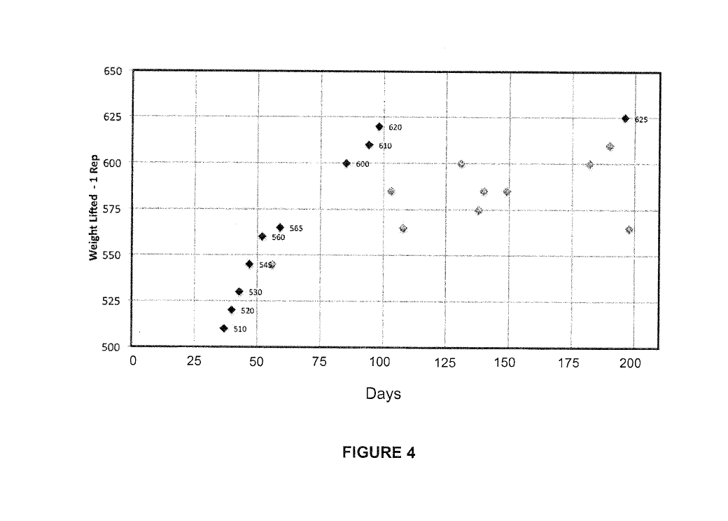

The subject completed 41 squat sessions out of 187 possible training days, averaging 1.5 squat sessions per week. 20 of those sessions worked up to a heavy single squat resulting in 10 new personal records. As shown in FIG. 4, the subject displayed a significant increase in squat performance over the 6-month period using the formulation.

### Bench Press

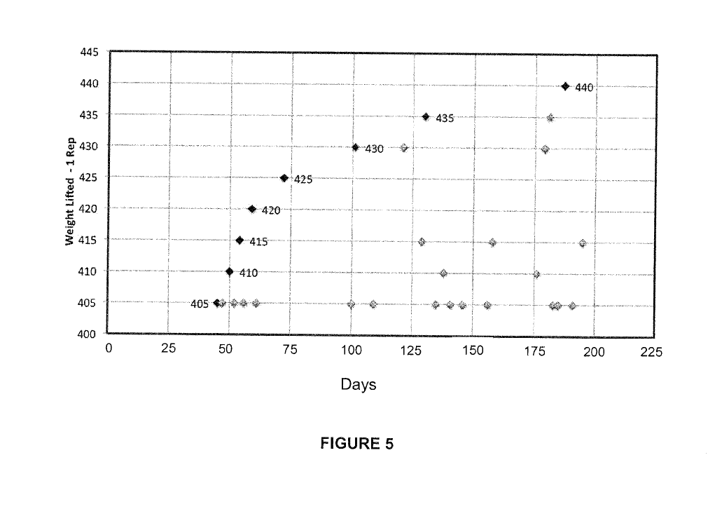

The subject completed 60 bench press sessions out of 187 possible training days averaging 2.2 bench press sessions per week. 29 of those sessions worked up to a heavy single lift resulting in 10 new personal records. As shown in FIG. 5, the subject displayed a significant increase in squat performance over the 6-month period using the formulation.

### Overall Effect on Performance and Recovery

The subject reported that the use of the formulation significantly improved recovery both from set-to-set during a training session and between training sessions relative to previous training without the use of the formulation.

The subject also reported that the improved recovery observed following use of the formulation allowed the subject to increase the number of training days in a month. Specifically, the subject reported being able to increase the number of heavy lifting training days by 20-30 more days over the study compared to previous 6-month training periods without the use of the formulation

The use of the formulation also resulted in a significant effect on peak power output demonstrated by the observed increase in peak single heavy lifts.

### EXAMPLE 4

The effects of the composition were assessed on a male 22 years of age, 205 pounds who had been power lifting for 2.5 years and competing at the national level

Each capsule contained 200 milligrams of citric acid, 300 milligrams of malic acid, 200 milligrams of pyruvic acid and 100 milligrams of ascorbic acid.

The subject trained as a power lifter 4 times per week, for 2-3 hours per session. The subject consumed 5 capsules prior to training, 5 capsules during training and 5 capsules post-training. Subject had been using the composition for 1 month

### Results

The subject reported being less sore the day after training compared to previous training sessions without using the composition. The subject noted that recovery time was the biggest benefit of using the composition. Subject also reported a decrease in rest time between sets, an increase in work capacity and that his volume load (weight lifted) per week increasing at a faster rate than before. Subject reported a better recovery time, that he was able to lift heavy more often, and that he was now squatting, benching and deadlifting close to double the amount of times per week that he was before using the capsules.

### EXAMPLE 5

Capsules containing 200 milligrams of citric acid, 300 milligrams of malic acid, 200 milligrams of pyruvic acid and 100 milligrams of ascorbic acid as described herein were tested in additional subjects engaged in lifting/power training.

Subject 1 consumed 5 capsules (4 grams) before exercise, 5 capsules during exercise and 5 capsules immediately post-exercise. Subject reported that the capsules kept him “recovered and physiologically “fresh” and that “(b)eing recovered with no residual fatigue allowed me to follow my plan and execute the prescribed volume and intensity for the day, continually increasing my performance and aiding me to continually progress with my training.”

Subject 2 consumed 5 capsules (4 grams) before exercise. Subject 2 reported “I have been training karate for 26 years and train 6 days a week now. With (the capsules) I get faster recovery, a higher level of strength and better energy. Also I increased my training time by about 60% and have no symptoms of delayed onset muscle soreness.”

Subject 3 consumed 3 capsules (2.4 grams) before exercise, 3 capsules (2.4 grams) during exercise, 3 capsules (2.4 grams) post exercise. Subject 3 reported “My goal is to best personal records and enjoy my time in the gym. (The capsules) helped me develop an edge I needed as an advanced athlete—increased endurance and recovery, less soreness in muscles, decreased rest time in between working sets, more explosive power.

Subject 4 consumed 4 capsules (3.2 grams) before exercise and 4 capsules (3.2 grams) post exercise. Subject 4 reported “I tend to train at moderate to high intensity always in the 65 to 85 percent of one rep max. My training goals are to continue to get stronger, stay injury free and add weight to my total each year indefinitely. When I take (the capsules), I find a sustained release of energy even though there are no stimulants in the product, I am able to train with consistent effort through a training session. My recovery isn't something I even consider when taking (the capsules) consistently, however I do notice soreness during recovery when I do not remember to take (the capsules).”

### EXAMPLE 6

Compositions containing 200 milligrams of citric acid, 300 milligrams of malic acid, 200 milligrams of pyruvic acid and 100 milligrams of ascorbic acid as described herein were tested in additional subjects under 3 conditions: (1) alone with water (2); with glucose; and (3) with proteinaceous formulations such as protein powder or amino acids.

Subjects were asked to exercise to their “lactic” threshold or beyond. The lactic threshold was defined the point at which muscles burn to the point where the athlete was no longer able to continue exercising.

The composition under condition 1 (alone with water) performed better than the composition under condition 2 (with glucose), and better than condition 3 (with proteinaceous formulations such as protein powder or amino acids).

The difference ranged from an incremental 20% to 50% in performance.

These in vivo results reflect experiments performed in vitro with cultured muscle cells, whereby depleting glycogen stores and refeeding with glucose resulted in an unhealthy cellular metabolic state likely due to increased mitochondrial stress.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### LIST OF REFERENCES

1. Armstrong R B, Warren G L, Warren J A. 1991. Mechanisms of exercise-induced muscle fiber injury. Sports Med. 12:184-207
2. Bendahan D, Mattei J P, Ghattas B, Confort-Gouny S, Le Guern M E, Cozzone P J. 2012. Citrulline/malate promotes aerobic energy production in human exercising muscle. Br J Sports Med. 36(4):282-9.
3. Benkhalifa M, Ferreira Y J, Chahine H, Louanjli N, Miron P, Merviel P, Copin H. 2014 Mitochondria: participation to infertility as source of energy and cause of senescence. Int J Biochem Cell Biol. 55:60-4.
4. Bruce M, Constantin-Teodosiu D, Greenhaff P L, Boobis L H, Williams C, Bowtell J L. 2001. Glutamine supplementation promotes anaplerosis but not oxidative energy delivery in human skeletal muscle. Am J Physiol Endocrinol Metab. 280(4):E669-75.
5. Chang C K, Chang Chien K M, Chang J H, Huang M H, Liang Y C, Liu T H. 2015. Branched-chain amino acids and arginine improve performance in two consecutive days of simulated handball games in male and female athletes: a randomized trial. PLoS One. 10(3):e0121866.
6. Currell K, Syed A, Dziedzic C E, King D S, Spriet L L, Collins J, Castell L M, Stear S J, Burke L M. 2010. A-Z of nutritional supplements: dietary supplements, sports nutrition foods and ergogenic aids for health and performance—part 12. Br J Sports Med. 44(12):905-7
7. Dabidi Roshan V, Babaei H, Hosseinzadeh M, Arendt-Nielsen L. 2013. The effect of creatine supplementation on muscle fatigue and physiological indices following intermittent swimming bouts. J Sports Med Phys Fitness 53(3):232-9.
8. Egras A M, Hamilton W R, Lenz T L, Monaghan M S. 2011. An evidence-based review of fat modifying supplemental weight loss products. J Obes. 2011. pii: 297315.
9. Fouré A, Nosaka K, Gastaldi M, Mattei J P, Boudinet H, Guye M, Vilmen C, Le Fur Y, Bendahan D, Gondin J. 2015. Effects of branched-chain amino acids supplementation on both plasma amino acids concentration and muscle energetics changes resulting from muscle damage: A randomized placebo controlled trial. Clin Nutr. pii: S0261-5614(15)00089-8.
10. Heinonen O J. 1996. Carnitine and physical exercise. Sports Med. 22(2):109-32.
11. Herda T J, Ryan E D, Stout J R, Cramer J T. 2008. Effects of a supplement designed to increase ATP levels on muscle strength, power output, and endurance. J Int Soc Sports Nutr. 5:3.
12. Hodgson A B(1), Randell R K, Boon N, Garczarek U, Mela D J, Jeukendrup A E, Jacobs D M. 2013. Metabolic response to green tea extract during rest and moderate-intensity exercise. J Nutr Biochem. 24(1):325-34.
13. Ishikura K, Miyazaki T, Ra S G, Endo S, Nakamura Y, Matsuzaka T, Miyakawa S, Ohmori H. 2011. Effect of taurine supplementation on the alterations in amino Acid content in skeletal muscle with exercise in rat. J Sports Sci Med. 10(2):306-14.
14. Ivy J L. 1998. Effect of pyruvate and dihydroxyacetone on metabolism and aerobic endurance capacity. Med Sci Sports Exerc. 30(6):837-43.
15. Jäger R, Metzger J, Lautmann K, Shushakov V, Purpura M, Geiss K R, Maassen N. 2008. The effects of creatine pyruvate and creatine citrate on performance during high intensity exercise. J Int Soc Sports Nutr. 5:4.
16. Kalman D, Colker C M, Stark R, Minsch A, Wilets I, Antonio J. 1999. Effect of pyruvate supplementation on body composition and mood. Current Therapeutic Research. 59(11):793-802.
17. Manjarrez-Montes de Oca R, Farfán-González F, Camarillo-Romero S, Tlatempa-Sotelo P, Francisco-Argüelles C, Kormanowski A, González-Gallego J, Alvear-Ordenes I. 2013. Effects of creatine supplementation in taekwondo practitioners. Nutr Hosp. 28(2):391-9.
18. Merante F, Mickle D A, Weisel R D, Li R K, Tumiati L C, Rao V, Williams W G, Robinson B H. 1998. Myocardial aerobic metabolism is impaired in a cell culture model of cyanotic heart disease. Am J Physiol. 275(5 Pt 2):H1673-81
19. Myers J, Ashley E. 1997. Dangerous curves. A perspective on exercise, lactate, and the anaerobic threshold. Chest. 111(3):787-95.
20. Newhame D J. 1988. The consequences of eccentric contractions and their relationship to delay onset muscle pain. Eur J Appl Physiol. 57:353-59
21. Parthimos T(1), Tsopanakis C, Angelogianni P, Schulpis K H, Parthimos N, Tsakiris S. 2007. L-cysteine supplementation prevents exercise-induced alterations in human erythrocyte membrane acetylcholinesterase and Na+,K+-ATPase activities. Clin Chem Lab Med. 45(1):67-72.
22. Pérez-Guisado J, Jakeman P M. 2010. Citrulline malate enhances athletic anaerobic performance and relieves muscle soreness. J Strength Cond Res. 24(5): 1215-22.
23. Powers H J, Bates C J, Eccles M, Brown H, George E. 1987. Bicycling performance in Gambian children: effects of supplements of riboflavin or ascorbic acid. Hum Nutr Clin Nutr. 41(1):59-69.
24. Putman C T, Jones N L, Lands L C, Bragg T M, Hollidge-Horvat M G, Heigenhauser G J. 1995. Skeletal muscle pyruvate dehydrogenase activity during maximal exercise in humans. Am J Physiol. 269(3 Pt 1):E458-68.
25. Rosa E F, Ribeiro R F, Pereira F M, Freymüller E, Aboulafia J, Nouailhetas V L. 2009. Vitamin C and E supplementation prevents mitochondrial damage of ileum myocytes caused by intense and exhaustive exercise training. J Appl Physiol 107(5):1532-8.
26. Russell C, Papadopoulos E, Mezil Y, Wells G D, Plyley M J, Greenway M, Klentrou P. 2014. Acute versus chronic supplementation of sodium citrate on 200 m performance in adolescent swimmers. J Int Soc Sports Nutr. 11:26.
27. Shan L, Wang B, Gao G, Cao W, Zhang Y. 2013. L-Arginine supplementation improves antioxidant defenses through L-arginine/nitric oxide pathways in exercised rats. J Appl Physiol (1985). 115(8):1146-55.
28. Sahlin K, Mogensen M, Bagger M, FernstrOm M, Pedersen P K. 2007. The potential for mitochondrial fat oxidation in human skeletal muscle influences whole body fat oxidation during low-intensity exercise. Am J Physiol Endocrinol Metab. 292(1):E223-30.

29. She P, Zhou Y, Zhang Z, Griffin K, Gowda K, Lynch C J. 2010. Disruption of BCAA metabolism in mice impairs exercise metabolism and endurance. J Appl Physiol (1985). 108(4):941-9.
30. Stanko R T, Tietze D L, Arch J E. 1992. Body composition, energy utilization, and nitrogen metabolism with a 4.25-MJ/d low-energy diet supplemented with pyruvate. American Journal of Clinical Nutrition. 56(4):630-635.
31. Stone M H, Sanborn K, Smith L L, O'Bryant H S, Hoke T, Utter A C, Johnson R L, Boros R, Hruby J, Pierce K C, Stone M E, Garner B. Effects of in-season (5 weeks) creatine and pyruvate supplementation on anaerobic performance and body composition in American football players. Int J Sport Nutr. 9(2):146-65.
32. Takeda K, Machida M, Kohara A, Omi N, Takemasa T. 2011. Effects of citrulline supplementation on fatigue and exercise performance in mice. J Nutr Sci Vitaminol (Tokyo). 57(3):246-50.
33. Tang F C, Chan C C, Kuo P L. 2014. Contribution of creatine to protein homeostasis in athletes after endurance and sprint running. Eur J Nutr. 53(1):61-71.
34. Timpmann S, Burk A, Medijainen L, Tamm M, Kreegipuu K, Vähi M, Unt E, Oöpik V.2012. Dietary sodium citrate supplementation enhances rehydration and recovery from rapid body mass loss in trained wrestlers. Appl Physiol Nutr Metab. 37(6):1028-37.
35. Tsakiris S, Karikas G A, Parthimos T, Tsakiris T, Bakogiannis C, Schulpis K H. 2009. Alpha-tocopherol supplementation prevents the exercise-induced reduction of serum paraoxonase 1/arylesterase activities in healthy individuals. EurJ Clin Nutr. 63(2):215-21.
36. Tyka A K, Chwastowski M, Cison T, Palka T, Tyka A, Szygula Z, Pilch W, Strzala M, Cepero M. 2015. Effect of creatine malate supplementation on physical performance, body composition and selected hormone levels in sprinters and long-distance runners. Acta Physiol Hung. 102(1):114-22.
37. Vukovich M D, Sharp R L, King D S, Kershishnik K. 1992. The effect of protein supplementation on lactate accumulation during submaximal and maximal exercise. Int J Sport Nutr. 2(4):307-16.
38. Wall B T, Stephens F B, Constantin-Teodosiu D, Marimuthu K, Macdonald I A, Greenhaff P L. 2011. Chronic oral ingestion of L-carnitine and carbohydrate increases muscle carnitine content and alters muscle fuel metabolism during exercise in humans. J Physiol. 589(Pt 4):963-73.
39. Wax B, Kavazis A N, Luckett W. 2015a. Effects of Supplemental Citrulline-Malate Ingestion on Blood Lactate, Cardiovascular Dynamics, and Resistance Exercise Performance in Trained Males. J Diet Suppl. February 12.
40. Wax B, Kavazis A N, Weldon K, Sperlak J. 2015b. Effects of supplemental citrulline malate ingestion during repeated bouts of lower-body exercise in advanced weightlifters. J Strength Cond Res. 29(3):786-92.
41. Wu J L, Wu Q P, Huang J M, Chen R, Cai M, Tan J B. 2007. Effects of L-malate on physical stamina and activities of enzymes related to the malate-aspartate shuttle in liver of mice. Physiol Res. 56(2):213-20.
42. Yavuz H U, Turnagol H, Demirel A H. 2014. Pre-exercise arginine supplementation increases time to exhaustion in elite male wrestlers. Biol Sport. 31(3):187-91.
43. Psychogios et al. The human serum metabolome. PLoS One. 2011 Feb. 16; 6(2):e16957
44. Free Radical Biology and Medicine 89 (2015): 62-71)
45. J Appl Physiol 107: 1532-1538 (2009)
46. Methods Mol Biol. (2012) 837:63-72.
47. Clinica Chimica Acta 129(1983):359-364.
48. Methods Mol Biol. (2013); 979: 65-70.
49. Appl. Biochem. Biotech. 56(1996):49-57.
50. Free Radic Biol Med. (2014) October; 75:69-83)

1. A composition comprising citric acid, malic acid, pyruvic acid and ascorbic acid, or salts thereof.

2. The composition of claim 1, consisting essentially of citric acid, malic acid, pyruvic acid and ascorbic acid, or salts thereof.

3. The composition of claim 1, further comprising succinic acid.

4. The composition of claim 1, comprising 2 to 4 parts by weight of pyruvic acid, 2 to 8 parts by weight of citric acid and 3 to 6 parts by weight of malic acid.

5. The composition of claim 4, comprising from 0.5 to 4 parts by weight of ascorbic acid.

6. The composition of claim 1, comprising from 200 to 400 mg of pyruvic acid, from 200 to 800 mg of citric acid and from 300 to 600 mg of malic acid.

7. The composition of claim 6, comprising from 50 to 400 mg of ascorbic acid or a salt thereof.

8. The composition of claim 1, wherein the molar ratio of malic acid to pyruvic acid to citric acid to ascorbic acid is about 1:1:1:1.

9. The composition of claim 1, wherein the molar ratio of malic acid to pyruvic acid to citric acid to ascorbic acid is about 1.5:1:1:0.5.

10. The composition of claim 1, comprising about 200 mg of pyruvic acid, about 300 mg of malic acid, about 200 mg of citric acid and about 100 mg of ascorbic acid.

11. The composition of claim 1, further comprising one or more citric acid cycle precursors, vitamins, minerals, micronutrients, electrolytes, carbohydrates, amino acids, proteins, flavouring agents and/or excipients.

12. (canceled)

13. (canceled)

14. The composition of claim 1, wherein the composition is in the form of a liquid, a paste, a gel, a bar, a powder, a tablet or a capsule.

15. The composition of claim 14, wherein the tablet is a lozenge, a candy or a chewing gum.

16. The composition of claim 1, wherein the composition does not include carbohydrates and/or protein.

17. A method of improving physical recovery following exertion in a subject, comprising administering to the subject an effective amount of the composition of claim 1.

18. The method of claim 17 wherein the composition is administered prior to, during or after exertion by the subject.

19. The method of claim 18, wherein the composition is administered prior to, during and after exertion by the subject.

20. The method of claim 19, wherein the composition is administered within 30 minutes prior to exertion by the subject, during exertion by the subject and within 30 minutes after exertion by the subject.

21. A method of improving mitochondrial function in a subject in need thereof comprising administering to the subject an effective amount of the composition of claim 1.

22. A method of decreasing cellular lactic acid in a subject in need thereof comprising administering to the subject an effective amount of the composition of claim 1.

23.-29. (canceled)
